(19) United States
(12) Patent Application Publication
(10) Pub. No.: US 2024/0077479 A1
(43) Pub. Date: Mar. 7, 2024

(54) DETECTION SYSTEM AND METHOD FOR THE MIGRATING CELL

(71) Applicant: DeepBrain Tech. Inc, Taipei City (TW)

(72) Inventors: Han-Lin Wang, Taipei City (TW); Chia-Wei Chen, Taipei City (TW); Yao-Wen Liang, Taipei City (TW); Ting-Chun Lin, Taipei City (TW); Yun-Ting Kuo, Taipei City (TW); You-Yin Chen, Taipei City (TW); Yu-Chun Lo, Taipei City (TW); Ssu-Ju Li, Taipei City (TW); Ching-Wen Chang, Taipei City (TW); Yi-Chen Lin, Taipei City (TW)

(73) Assignee: DeepBrain Tech. Inc, Taipei City (TW)

(21) Appl. No.: 18/232,724

(22) Filed: Aug. 10, 2023

(30) Foreign Application Priority Data
Sep. 1, 2022 (TW) 111133264

(51) Int. Cl. G01N 33/553; A61B 5/00; B01L 3/00; G01N 33/543; G01N 33/574

(52) U.S. Cl. CPC G01N 33/553; A61B 5/0066; B01L 3/502761; G01N 33/54333; G01N 33/574; G01N 2021/0143

(57) ABSTRACT

A detection system and method for the migrating cell is provided. The system is configured to detect a migrating cell combined with an immunomagnetic bead. The system includes a platform, a microchannel, a magnetic field source, a coherent light source and an optical sensing module. The microchannel is configured to allow the migrating cell to flow in it along a flow direction. The magnetic field source is configured to provide magnetic force to the migrating cell combined with the immunomagnetic bead. The magnetic force includes at least one magnetic force component and the magnetic force component is opposite to the flow direction of the microchannel. The coherent light source is configured to provide the microchannel with the coherent light. The optical sensing module is configured to receive the interference light caused by the coherent light being reflected by the sample inside the microchannel.

### CROSS-REFERENCE TO RELATED APPLICATION

This non-provisional application claims priority under 35 U.S.C. § 119(a) to Patent Application No. 111133264 filed in Taiwan, R.O.C. on Sep. 1, 2022, the entire contents of which are hereby incorporated by reference.

### BACKGROUND

### Technical Field

This application relates to a detection system and method for a migrating cell, applying optics.

### Related Art

The metastasis of cancer is the leading cause of death, and it is closely related to migrating cells 104 shedding from tumor in situ 101, namely circulating tumor cells (CTCs). FIG. 1 is a schematic diagram of circulating tumor cells metastasizing with blood vessels, referring to FIG. 1. When a distal metastatic tumor 102 is found, it means that some tumor cells have spread from an original location of the tumor to other parts of the body via blood vessels 103. The invasion of the metastatic tumor is actively monitored to obtain more adequate information as much as possible, which is the key to determine a follow-up treatment plan. Generally, CTCs will travel to various organs and tissues via the blood and lymphatic system in the body, causing tumor metastasis. If CTCs can be detected and the accuracy of the detection can be improved, this detection can become one of keys to monitor cancer changes.

In recent years, the development of technologies at home and abroad has mainly focused on the development of detection and capture of CTCs, such that CTC detection has the characteristics of non-invasiveness, direct and accurate detection of tumor cells, real-time detection, monitoring of spread tumor cells in the blood, and early indication of cancer cell spread changes, that is, personalized medicine, such as prognosis, treatment monitoring and selection, is performed via CTC counting and molecular characterization analysis. However, since CTCs are sparse, they are difficult to detect, and the process involves many processing steps, the CTC detection technology still has many limitations in clinical application. At present, common CTC cell detection ways in the market include flow cytometry, immunofluorescence, fluorescence in situ hybridization (FISH), real-time polymerase chain reaction (RT-PCR) and next generation sequencing (NGS). For the flow cytometry, a large number of rapid detection schemes are provided, but due to its low detection sensitivity, a large number of samples are required and cell morphology cannot be observed. The immunofluorescence has the advantages that the cell morphology can be directly observed, the detection sensitivity is high and the speed is fast, but due to the diversity of the cell morphology, there will be subjective differences in judgment due to the heterogeneity of cell antigen expression. FISH provides molecular detection grades, and has the advantages of high stability, high sensitivity and high specificity, but its disadvantages are that a shorter probe has low hybridization efficiency and is susceptible to interference. Since RT-PCR can directly detect RNA of CTC, the sensitivity is high, but it is limited by the problems that RNA is prone to degradation, pollution and interference. NGS can be applied in a wide range of detections, and has high sensitivity and fast speed, but due to its high price, it cannot be popularized, and the cell morphology cannot be observed.

Each of the above detection methods has its own advantages and disadvantages. However, as for technologies currently used, a longer period of time is required for testing, and patients need to go to hospitals and clinics repeatedly, which is both troublesome and time-consuming for residents in some villages and towns lacking medical care. Moreover, the high cost of testing also weakens the willingness of the patients to undergo self-paying examinations, allowing their illnesses to spread. Furthermore, in some detection technologies, the number of CTCs cannot be accurately measured, resulting in low detection efficiency.

### SUMMARY

In view of this, this application proposes a detection system for a migrating cell. The detection system for the migrating cell, suitable for detecting a labeled sample, the labeled sample including a migrating cell and an immunomagnetic bead combined with the migrating cell, the immunomagnetic bead including a magnetic bead and an antibody embedded into the surface of the magnetic bead, the antibody being combined with a surface antigen of the migrating cell, and the detection system for the migrating cell including a platform; a microchannel, provided on the platform, and configured to allow the labeled sample to flow in it along a flow direction; a magnetic field source, provided outside the microchannel, and configured to provide a magnetic field to the microchannel, and the magnetic field applying a magnetic force to the magnetic bead of the labeled sample, and the magnetic force including at least one magnetic force component and the magnetic force component being opposite to the flow direction; a coherent light source, provided above the platform, and configured to apply coherent light to the microchannel; and an optical sensing module, provided above the platform, and configured to receive interference light caused by the coherent light being reflected by the labeled sample inside the microchannel.

According to some embodiments, the magnetic field source is a coil, and the coil surrounds the microchannel and is configured to provide the magnetic field.

According to some embodiments, the detection system further includes an image processing module, the image processing module calculating the flow velocity of the labeled sample in the microchannel according to the contrast of the interference light, and judging that the migrating cell passes through the microchannel when the value of the flow velocity produces a surge change.

According to some embodiments, the image processing module calculates the flow velocity of the labeled sample in the microchannel according to the following formula:

$$V ( i , j ) ∝ \frac{1}{T {K ( i , j )}^{2}}$$

Where V is flow velocity, T is exposure time, K is contrast, and i and j are pixel coordinates.

According to some embodiments, the pipe diameter of the microchannel is greater than or equal to 10 μm and less than or equal to 50 μm.

According to some embodiments, the wavelength of the coherent light is greater than or equal to 660 nm and less than or equal to 760 nm.

This application further provides a detection method for a migrating cell. The detection method for the migrating cell, configured to detect a migrating cell in a blood sample, the migrating cell including a surface antigen, and the detection method for the migrating cell including the following steps: mixing the blood sample with an immunomagnetic bead to form a labeled sample, the immunomagnetic bead including a magnetic bead and an antibody embedded into the surface of the magnetic bead, and the antibody being combined with the surface antigen of the migrating cell; allowing the labeled sample to pass through a microchannel in a flow direction; applying a magnetic field to the microchannel, the magnetic field applying a magnetic force to the magnetic bead of the labeled sample, and the magnetic force including at least one magnetic force component and the magnetic force component being opposite to the flow direction; applying coherent light to the microchannel; and receiving interference light caused by the coherent light being reflected by the labeled sample inside the microchannel.

According to some embodiments, after mixing the blood sample with the immunomagnetic bead, the method further includes: centrifugally separating and removing the immunomagnetic bead not combined with the migrating cell.

According to some embodiments, after the receiving the interference light caused by the coherent light being reflected by the labeled sample inside the microchannel, the method further includes: the applying a magnetic field to the microchannel to drain the labeled sample, and collecting the migrating cell combined with the immunomagnetic bead and attracted by the magnetic field.

According to some embodiments, the migrating cell is a circulating tumor cell, and an antibody of the immunomagnetic bead is an EpCAM antibody or Her2 antibody.

### BRIEF DESCRIPTION OF THE DRAWINGS

FIG. 1 is a schematic diagram of circulating tumor cells metastasizing with blood vessels;

FIG. 2 is a flowchart of a detection method for a migrating cell according to some embodiments in this application;

FIG. 3 is a schematic diagram of an immunomagnetic bead according to some embodiments in this application;

FIG. 4 is a schematic diagram of centrifugal separation of free immunomagnetic beads according to some embodiments in this application;

FIG. 5 is a schematic diagram of a detection system for a migrating cell according to some embodiments in this application;

FIG. 6 is a schematic diagram of a laser spot principle according to some embodiments in this application;

FIG. 7 is an actual shooting picture of a laser spot according to some embodiments in this application;

FIG. 8 is a schematic diagram of decelerating a microchannel with a magnetic field according to some embodiments in this application;

FIG. 9A is a schematic diagram of a labeled sample not flowing according to some embodiments in this application;

FIG. 9B is a schematic diagram of a labeled sample flowing according to some embodiments in this application;

FIG. 10 is a schematic diagram of surge changes in flow velocity according to some embodiments in this application; and

FIG. 11 is an absorption spectrum of conventional hemoglobin.

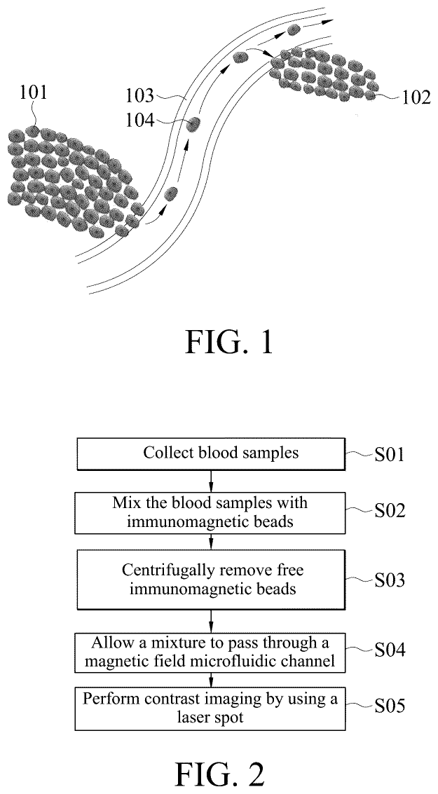

### DETAILED DESCRIPTION

FIG. 2 is a flowchart of a detection method for a migrating cell according to some embodiments in this application, referring to FIG. 2. According to some embodiments, the detection method for a migrating cell 104 can be applicable to operation procedures of laboratories or medical units, or can be applicable to operation procedures of research or medical devices. The detection method for the migrating cell 104 may include collecting a blood sample 108 from a subject (step S01), for example collecting blood from a blood vessel 103 of the subject or capturing the blood sample 108 from a test tube. The blood sample 108 may be original blood or blood in which some molecules, proteins or blood cells are filtered out, and may at least include the migrating cell 104 to be detected. The migrating cell 104 may be, but not limited to, cells originating from the subject itself and present in the blood vessel 103 of the subject, such as blood cells, red blood cells 105, and CTCs, or substances originating from the outside and present in the blood vessel 103 of the subject, such as bacteria, mycetes, and viruses. The migrating cell 104 may include surface antigens present on cell membranes, cell walls, flagella or protein coat surfaces, for example, surface antigens such as an epithelial cell adhesion molecule (EpCAM) on the surface of CTC, cytokeratin, mammaglobulin (MGB), thyroid transcription factor-1 (TTF-1), prostate-specific membrane antigen (PSMA), or human epithelial growth factor receptor 2 (HER2).

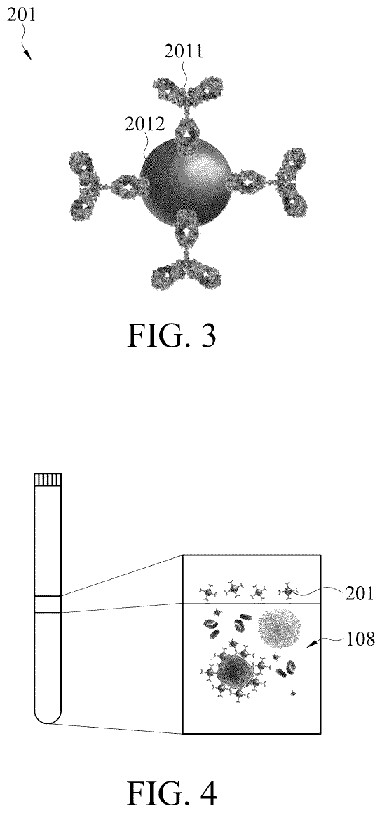

In the detection method for the migrating cell 104, the blood sample 108 is mixed with the immunomagnetic bead 201 to form the labeled sample (step S02). FIG. 3 is a schematic diagram of an immunomagnetic bead according to some embodiments in this application, referring to FIG. 3. The immunomagnetic bead 201 includes a magnetic bead 2012 and an antibody 2011 embedded into the surface of a magnetic bead 2012. The antibody 2011 can be combined with the surface antigen of the migrating cell 104. The magnetic bead 2012 can include an iron oxide magnetic core and a coating. The material of the magnetic core may be, but not limited to, magnetic substances such as Fe₃O₄, Fe₂O₃ or the like. The material of the coating may be, but not limited to, SiO₂, polyvinyl alcohol, glucan, agarose, agarose gel or polystyrene to provide biological compatibility and protect the magnetic core. According to some embodiments, the diameter of the magnetic bead 2012 may be between 50 nm and 5 μm. The immunomagnetic bead 201 is combined with the surface antigen of the migrating cell 104 due to the antibody 2011, so that the labeled migrating cell 104 has magnetic susceptibility. According to some embodiments, in order to remove the immunomagnetic bead 201 not combined with the migrating cell 104, the labeled sample is allowed to pass through a filter membrane to remove free immunomagnetic beads 201. According to some embodiments, in order to remove the immunomagnetic bead 201 not combined with the migrating cell 104, the immunomagnetic bead 201 not combined with the migrating cell 104 is centrifugally separated and removed (step S03). FIG. 4 is a schematic diagram of centrifugal separation of free immunomagnetic beads according to some embodiments in this application, referring to FIG. 4. By way of examples, the labeled sample is processed with a centrifugal machine, where due to the small mass, the immunomagnetic bead 201 not combined with the migrating cell 104 is suspended on the surface layer of a test tube, while other blood cells or migrating cells 104 combined with the immunomagnetic beads 201 are precipitated due to their large mass. Here, after removing the free immunomagnetic bead 201, a substance with the magnetic susceptibility in the labeled sample is only the immunomagnetic bead 201 combined with the migrating cell 104.

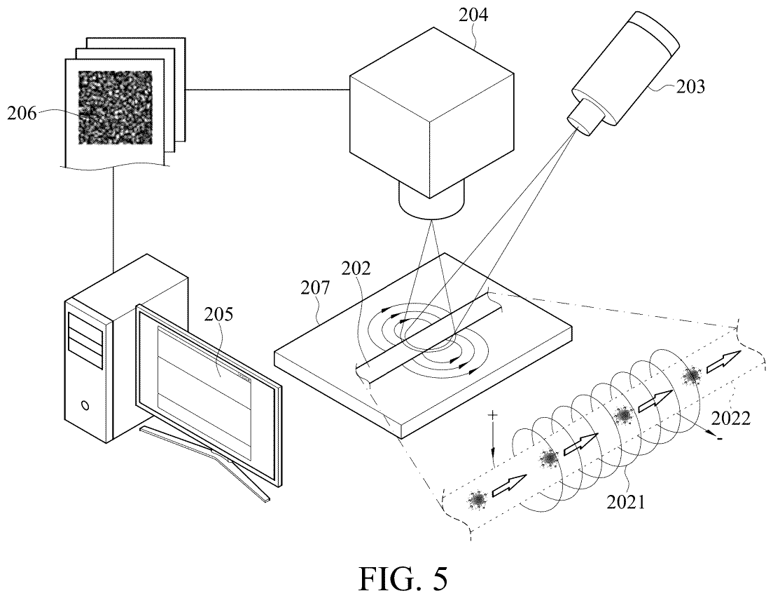

In the detection method for the migrating cell 104, the mixture of the labeled samples is allowed to pass through a magnetic field microchannel 202 in a flow direction (step S04). FIG. 5 is a schematic diagram of a detection system for a migrating cell according to some embodiments in this application, referring to FIG. 5. According to some embodiments, the detection system for the migrating cell 104 includes a platform 207, a microchannel 202, a coherent light source 203 and an optical sensing module 204. The platform 207 is configured to carry other elements for these elements to be directly or indirectly fixed or placed thereon. According to some embodiments, the platform 207 may be a plate of an anti-vibration table or a detection machine to provide other elements with a stable operating environment.

The magnetic field microchannel 202 is provided on the platform 207, and includes a microchannel 2022 and a magnetic field source 2021. According to some embodiments, the microchannel 2022 is directly or indirectly fixed to the platform 207. By way of examples, the microchannel 2022 is fixed to a fixture of the platform 207 or placed in a preinstalled holding space. According to some embodiments, the magnetic field source 2021 is directly or indirectly fixed to the platform 207. The microchannel 2022 is configured to allow the labeled sample to flow in it along a flow direction. According to some embodiments, the width of the microchannel 2022 only allows one to several migrating cells 104 to pass through to provide good counting and detection conditions, as explained in detail later. The surface of the microchannel 2022 is sufficiently transparent, which is enough for coherent light L1 to penetrate and be sensed by optical sensing module 204. By way of examples, the microchannel 2022 is made of silicon dioxide, quartz, silicon crystal, polymethyl methacrylate, polydimethylsiloxane, polystyrene or polycarbonate. According to some embodiments, the microchannel 2022 can push or pull the labeled sample by using a vacuum pump or peristaltic pump, or drive the flow of the microchannel 2022 by employing gravity, concentration gradient, electrical potential difference and other ways. The magnetic field source 2021 may be a magnet, an electromagnet, or a coil. The magnetic field source 2021 is provided outside the microchannel 2022, and configured to provide a magnetic field to the microchannel 2022, and the magnetic field applies a magnetic force to the immunomagnetic bead 201 of the labeled sample. The magnetic force includes at least one magnetic force component and the magnetic force component is opposite to the flow direction of the labeled sample inside the microchannel 2022. According to some embodiments, the magnetic field source 2021 employs an electromagnet or coil energized with direct current to provide a stable magnetic field direction B, ensuring that the magnetic field direction B and a flow direction F of the microchannel 2022 keep a fixed relative relationship to avoid turbulence inside the microchannel 2022. According to some embodiments, the magnetic field source 2021 is the coil surrounding the microchannel 2022. In this way, a uniform magnetic field provided by the coil avoids that turbulence is caused by different flow velocity at different positions inside the microchannel 2022, thereby achieving the effect that the migrating cells 104 gradually pass through. The magnetic force component may refer to a projection on a magnetic force vector vs. a flow direction vector of the microchannel 2022. By way of examples, if the flow direction vector of the microchannel 2022 is (1,0) and the magnetic force vector is (−3, −4), the angle therebetween is 233°, and the magnetic force includes a magnetic field component (−3,0) opposite to the flow direction vector (1,0) of the microchannel 2022. In this way, the immunomagnetic bead 201 with magnetic susceptibility and its labeled migrating cell 104, when flowing through the magnetic field, are subjected to magnetic force and decelerate.

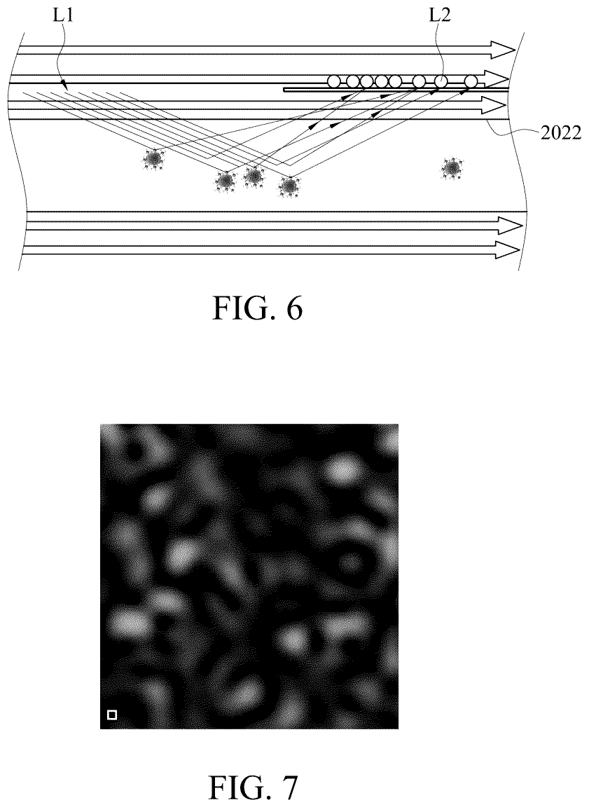

In the detection method for the migrating cell 104, the coherent light L1 is applied to the microchannel 2022 and the interference light caused by the coherent light L1 being reflected by the labeled sample inside the microchannel 2022 is received (step S05). According to some embodiments, the coherent light source 203 used for emitting the coherent light L1 may be a laser light source, for example a red He—Ne laser with a wavelength of 632 nm or a green laser with a wavelength of 532 nm. The coherent light source 203 is provided above the platform 207. According to some embodiments, the coherent light source 203 is directly or indirectly fixed to the platform 207. By way of examples, it is provided on a wall surface directly connected to or a top surface indirectly connected to the plate of the detection machine. Preferably, the coherent light source 203 may employ red laser light with a wavelength greater than or equal to 650 nm and less than or equal to 720 nm to match the absorbance of hemoglobin, as explained in detail later. FIG. 6 is a schematic diagram of a laser spot principle according to some embodiments in this application, referring to FIG. 6. The labeled sample inside the microchannel 2022 includes blood cells, migrating cells 104, and blood solutes scattered therein, so that the coherent light L1 is reflected after being incident upon the microchannel 2022. The reflected coherent light L1 generates different degrees of constructive interference light L2 in different spatial positions. The constructive interference light is received by the optical sensing module 204 to generate a spot image 206. According to some embodiments, the optical sensing module 204 may be a camera. The optical sensing module 204 can be directly or indirectly fixed to the platform 207. By way of examples, it is provided on a wall surface directly connected to or a top surface indirectly connected to the plate of the detection machine. FIG. 7 is an actual shooting picture of a laser spot according to some embodiments in this application, referring to FIG. 7. The optical sensing module 204 is suitable for sensing the constructive interference light L2 caused by reflection of the coherent light L1 emitted by the coherent light source 203, resulting in a one-dimensional spot image 206 or a two-dimensional spot image 206 as shown in FIG. 7. The bright part of FIG. 7 is formed by the constructive interference light L2, while the dark part is formed by destructive interference light.

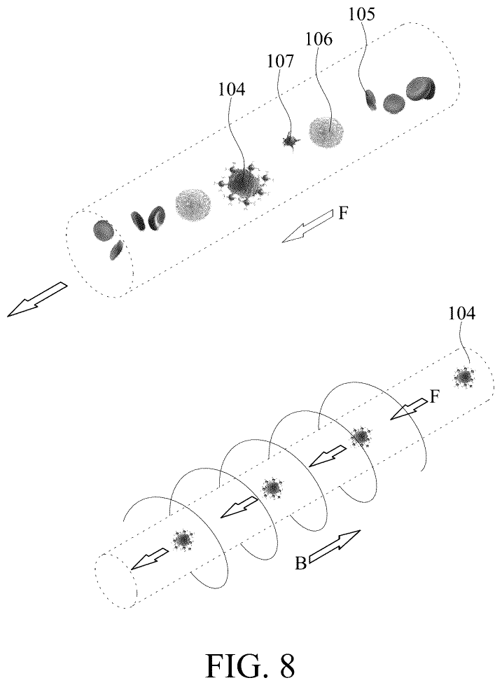

When the labeled sample inside the microchannel 2022 begins to flow, the spot undergoes a light and dark change. For pixels in fixed positions, within a pixel range as box-selected in the bottom left corner of FIG. 7, the slower the flow velocity of the labeled sample is, the slower the change speed of the spot is, and the larger the standard deviation of a brightness value within the pixel range is. On the contrary, the faster the flow velocity of the labeled sample is, the faster the change speed of the spot is, and the smaller the standard deviation of the brightness value within the pixel range is. By way of examples, in the same period of 1 second, 10 flashes are received within the pixel range when the flow velocity is slow, and 500 flashes are received within the pixel range when the flow velocity is fast. In this way, for images within the same pixel range, multiple flashes per unit time makes a sampling value averaged to obtain almost identical values, resulting in lower standard deviation and contrast (the contrast is represented as the standard deviation/average intensity per unit time). This phenomenon can be described by the following formula:

$$V ( i , j ) ∝ \frac{1}{T {K ( i , j )}^{2}}$$

Where V is the flow velocity, T is exposure time, K is the contrast, and i and j are pixel coordinates of a two-dimensional image. Therefore, the faster the flow velocity of the labeled sample per unit exposure time is, the smaller the measured spot contrast is. Conversely, an image processing module 205 receives the spot image 206 sensed by the optical sensing module 204. When the image processing module 205 judges that the spot contrast suddenly increases, representing that the flow velocity of the labeled sample inside the microchannel 2022 suddenly decreases. FIG. 8 is a schematic diagram of decelerating a microchannel with a magnetic field according to some embodiments in this application, referring to FIG. 8. The labeled sample is placed in the microchannel 2022, and is allowed to flow in the flow direction F. The labeled samples may include the migrating cells 104, the red blood cells 105, white blood cells 106 and platelets 107, where the red blood cells 105 account for the highest proportion and the migrating cells 104 account for a small amount. When the magnetic field opposite to its flow direction F is applied to the labeled sample, the migrating cell 104 labeled with the immunomagnetic bead 201 therein will be decelerated, thereby influencing the overall flow velocity of the labeled sample, and the contrast change in the spot image 206. According to some embodiments, in order to optimize the deceleration effect of the labeled sample and improve the ability to count the migrating cells 104, the pipe diameter of the microchannel 2022 is set to be greater than or equal to 10 μm and less than or equal to 50 μm. In general, the diameter of CTC is about 10-20 μm, the diameter of the red blood cell 105 is 6-8 μm, and the diameter of the white blood cell 106 is 10-15 μm. Therefore, the pipe diameter of the microchannel 2022 needs to be at least greater than 10 μm to facilitate the migrating cells 104 or blood cells to pass through, and less than or equal to 50 μm to limit the number of the passing migrating cells 104 or blood cells to be about 1 to 3. In this way, the migrating cells 104 or blood cells sequentially pass through the microchannel 2022, without a large number of migrating cells 104 or blood cells passing through at once, which is beneficial for cell counting. Moreover, when the migrating cell 104 labeled with the immunomagnetic bead 201 is decelerated due to magnetic field interference, other blood cells are blocked and unable or difficult to pass through quickly by the decelerated migrating cells 104, thus decreasing the overall flow velocity of the labeled sample at the same time, and reflecting the change in the spot image 206.

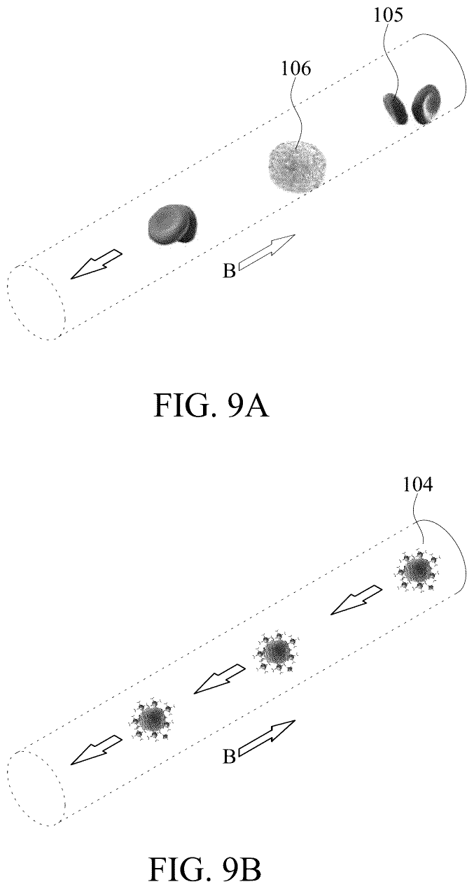

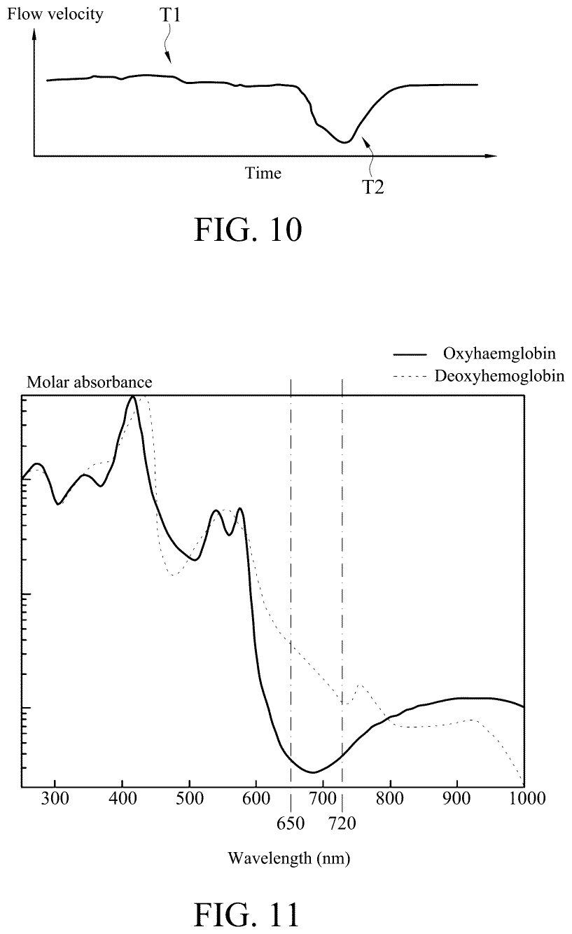

FIG. 9A is a schematic diagram of a labeled sample not flowing according to some embodiments in this application; FIG. 9B is a schematic diagram of a labeled sample flowing according to some embodiments in this application; and FIG. 10 is a schematic diagram of surge changes in flow velocity according to some embodiments in this application, referring to FIGS. 9A, 9B and 10 together. The horizontal axis of FIG. 10 presents time, while the vertical axis presents the flow velocity of the labeled sample. FIG. 9A presents the state of the microchannel 2022 at time point T1. At this time, only the white blood cell 106 and the red blood cell 105 without magnetic susceptibility pass through the microchannel 2022, and thus, the overall flow velocity of the labeled sample is not influenced by the magnetic field. Here, the spot image 206 presents a lower contrast and represents a higher overall flow velocity. FIG. 9B presents the state of the microchannel 2022 at time point T2. At this time, the migrating cells 104 labeled with the immunomagnetic beads 201 with the magnetic susceptibility pass through the microchannel 2022. Since the labeled migrating cell 104 is decelerated by the magnetic field to block the microchannel 2022, the overall flow velocity of the labeled sample is influenced. Here, the spot image 206 presents a higher contrast and represents a lower overall flow velocity. Since the migrating cells 104 account for a minority of the labeled samples, most of the time during the test period, the labeled samples present the flow velocity of time point T1 as shown in FIG. 10. However, when one to several migrating cells 104 labeled with the immunomagnetic beads 201 pass through, influenced by the magnetic field, the labeled samples will instantaneously present a lower flow velocity, resulting in the surge labeled at time point T2 in FIG. 10. In this way, the number of the migrating cells 104 can be estimated by calculating the number of times of surges, thereby achieving the effects of high sensitivity and rapid detection. In view of the foregoing, according to some embodiments, the detection system for the migrating cell 104 includes an image processing module 205. The image processing module 205 calculates the flow velocity of the labeled sample according to the contrast of the spot image 206, and calculates the number of the passing migrating cells 104 according to the number of times of surges of the flow velocity of the labeled sample.

According to some embodiments, since the labeled sample is mostly composed of the red blood cells 105, the leading cause of the spot image 206 is the red blood cells 105. That is, in the detection method for the migrating cell 104, the migrating cell 104 labeled with the immunomagnetic bead 201 is decelerated by using the magnetic field, and the red blood cell 105 in the labeled sample is blocked and decelerated at the same time. The optical sensing module 204 detects the constructive interference light L2 caused by the coherent light L1 being reflected by a large number of red blood cells 105, thereby obtaining the spot image 206. From this point of view, the absorbance of the red blood cell 105 to the coherent light L1 has a key influence on imaging of the spot image 206. FIG. 11 is an absorption spectrum of conventional hemoglobin, referring to FIG. 11. The horizontal axis of FIG. 11 is the wavelength of absorbed light, the vertical axis is molar absorbance, the solid line presents the change of absorbance of oxyhaemglobin, and the dashed line presents the change of absorbance of deoxyhemoglobin. The oxyhaemglobin and the deoxyhemoglobin have the highest absorbance for light at around 400 nm. The absorbance of the oxyhaemglobin for light 590 nm or above sharply decreases, while the absorbance for light between 650 nm and 720 nm is significantly lower than other wave ranges. Conversely, the oxyhaemglobin has the highest reflectivity to light from 650 nm to 720 nm. Therefore, according to some embodiments, the wavelength of the coherent light L1 caused by the coherent light source 203 is set to be greater than or equal to 650 nm and less than or equal to 720 nm, so that the optical sensing module 204 receives interference light with higher intensity, thereby improving the ability to distinguish signal contrast.

According to some embodiments, a cell capture module can be further attached to the microchannel 2022, and the cell capture module may include a magnetic field source and a cell collection region. The magnetic field source of the cell capture module may be, but not limited to, a magnet, an electromagnet or a coil. The magnetic field source of the cell capture module can be placed or fixed around the microchannel 2022. The cell collection region is configured to collect the migrating cells 104, and the cell collection region may be a holding space inside the microchannel 2022 or an external container. After the migrating cells 104 pass through the magnetic field microchannel 202 and counting is completed, the magnetic field generated by the magnetic field source of the cell capture module attracts these migrating cells 104 labeled with the immunomagnetic beads 201 and the migrating cells are drained to the cell collection region. After that, the cell morphology can be further judged with a cell morphology detection method. In this way, in the detection method for the migrating cell 104, a rapid and accurate preliminary assessment is firstly performed on the content of the migrating cells 104 in a specimen. When it is judged that the content is abnormal, these migrating cells 104 are then captured to further determine the cell morphology, and the detection speed and accuracy are balanced by graded detection.

In summary, in the process of cancer treatment, the most daunting is often the metastasis of cancer, which if not detected early, the recurrence rate and mortality rate of cancer will be significantly increased. If the cancer metastasis is detected and treated early, the subsequent risk caused by the cancer metastasis can be greatly reduced. Therefore, providing fast, accurate, affordable, and widely available early screening systems has become a major development focus for cancer treatment. In the detection method for the migrating cell 104 in this application, counting detection of single cells is performed by applying the microchannel 2022, and thus, the detection can be completed by only a very small amount of blood samples 108. The applicant finds from the study that the detection method for the migrating cell 104 can be performed only by drawing 8-10 ml of whole blood. Furthermore, the microchannel 2022 can be manufactured in mass through chip processing, thereby effectively reducing production costs. Compared with a traditional way for directly performing comprehensive cell number and cell morphology assessment on the whole blood, the detection method for the migrating cell 104 provides a rapid screening scheme: firstly, specimens of low-risk patients with lower content of migrating cells 104 are screened and excluded by using cell counting, and then samples of the migrating cells 104 in the specimens of high-risk patients are provided for further standardized judgment by using a traditional detection device. Overall, the detection method for the migrating cell 104 improves the detection efficiency and allows for widespread rapid screening for a large number of early cancer patients, thereby reducing the prevalence of metastatic cancer.

Although the application has been described in considerable detail with reference to certain preferred embodiments thereof, the disclosure is not for limiting the scope of the application. Persons having ordinary skill in the art may make various modifications and changes without departing from the scope and spirit of the application. Therefore, the scope of the appended claims should not be limited to the description of the preferred embodiments described above.

What is claimed is:

1. A detection system for a migrating cell, suitable for detecting a labeled sample, the labeled sample comprising a migrating cell and an immunomagnetic bead combined with the migrating cell, the immunomagnetic bead comprising a magnetic bead and an antibody embedded into the surface of the magnetic bead, the antibody being combined with a surface antigen of the migrating cell, and the detection system for the migrating cell comprising:
a platform;
a microchannel, provided on the platform, and configured to allow the labeled sample to flow in it along a flow direction;
a magnetic field source, provided outside the microchannel, and configured to provide a magnetic field to the microchannel, and the magnetic field applying a magnetic force to the magnetic bead of the labeled sample, and the magnetic force comprising at least one magnetic force component and the magnetic force component being opposite to the flow direction;
a coherent light source, provided above the platform, and configured to apply coherent light to the microchannel; and
an optical sensing module, provided above the platform, and configured to receive interference light caused by the coherent light being reflected by the labeled sample inside the microchannel.

2. The detection system according to claim 1, wherein the magnetic field source is a coil, and the coil surrounds the microchannel and is configured to provide the magnetic field.

3. The detection system according to claim 2, further comprising an image processing module, the image processing module calculating the flow velocity of the labeled sample in the microchannel according to the contrast of the interference light, and judging that the migrating cell passes through the microchannel when the value of the flow velocity produces a surge change.

4. The detection system according to claim 3, wherein the image processing module calculates the flow velocity of the labeled sample in the microchannel according to the following formula:
$$V ( i , j ) ∝ \frac{1}{T {K ( i , j )}^{2}}$$
wherein V is flow velocity, T is exposure time, K is contrast, and i and j are pixel coordinates.

5. The detection system according to claim 1, wherein the pipe diameter of the microchannel is greater than or equal to 10 μm and less than or equal to 50 μm.

6. The detection system according to claim 1, wherein the wavelength of the coherent light is greater than or equal to 650 nm and less than or equal to 720 nm.

7. A detection method for a migrating cell, configured to detect a migrating cell in a blood sample, the migrating cell comprising a surface antigen, and the detection method for the migrating cell comprising the following steps:
mixing the blood sample with an immunomagnetic bead to form a labeled sample, the immunomagnetic bead comprising a magnetic bead and an antibody embedded into the surface of the magnetic bead, and the antibody being combined with the surface antigen of the migrating cell;
allowing the labeled sample to pass through a microchannel in a flow direction;
applying a magnetic field to the microchannel, the magnetic field applying a magnetic force to the magnetic bead of the labeled sample, and the magnetic force comprising at least one magnetic force component and the magnetic force component being opposite to the flow direction;
applying coherent light to the microchannel; and
receiving interference light caused by the coherent light being reflected by the labeled sample inside the microchannel.

8. The detection method according to claim 7, after mixing the blood sample with the immunomagnetic bead, further comprising:
centrifugally separating and removing the immunomagnetic bead not combined with the migrating cell.

9. The detection method according to claim 8, after the receiving the interference light caused by the coherent light being reflected by the labeled sample inside the microchannel, further comprising:
the applying a magnetic field to the microchannel to drain the labeled sample, and collecting the migrating cell combined with the immunomagnetic bead and attracted by the magnetic field.

10. The detection method according to claim 7, wherein the migrating cell is a circulating tumor cell, and an antibody of the immunomagnetic bead is an EpCAM antibody or Her2 antibody.
